SEQUENCE LISTING (2 sequences)

SEQ ID NO: 1 - protein, 128 aa, Artificial Sequence
  Human-mouse - transgenic construct HuZAF VL (mature light chain begins at amino acid 21)
METDTLLLWVLLLWVPGSTGDIQMTQSPSTLSASVGDRVTITCKASENVDTYVSWYQQKPGKAPKLLIYGASNRYTGVPSRFSGSGSGTDFTLTISSLQPDDFATYYCGQSYNYPFTFGQGTKVEVKR

SEQ ID NO: 2 - protein, 136 aa, Artificial Sequence
  Human-mouse - transgenic construct HuZAF VH (mature heavy chain begins at amino acid 20)
MGWSWIFLFLLSGTAGVHSQVQLVQSGAELKKPGSSVKVSCKASGYIFTSSWINWVKQAPGQGLEWIGRIDPSDGEVHYNQDFKDKATLTVDKSTNTAYMELSSLRSEDTAVYYCARGFLPWFADWGQGTLVTVSS

(19) United States
(12) Patent Application Publication
(10) Pub. No.: US 2004/0052791 A1
(43) Pub. Date: Mar. 18, 2004

(54) Treatment of Crohn's disease or psoriasis using anti-interferon gamma antibodies

(76) Inventors: Rolf Ehrhardt, (US); Dan Levitt, San Francisco, CA (US); Beth Layug, San Francisco, CA (US); Donald O'Neill, Furlong, PA (US); Nancy Wedel, Oakland, CA (US); Lars Ostberg, Los Altos, CA (US)

Correspondence Address: HOWREY SIMON ARNOLD & WHITE, LLP, BOX 34, 301 RAVENSWOOD AVE., MENLO PARK, CA, 94025, US

(21) Appl. No.: 10/440,202

(22) Filed: May 16, 2003

Related U.S. Application Data
(60) Provisional application No. 60/383,310, filed on May 22, 2002.

(51) Int. Cl. A61K 39/395

(52) U.S. Cl. 424/145.1

(57) ABSTRACT

The present invention provides a method of treating autoimmune diseases. In particular, it provides a method for the treatment of Crohn's disease or psoriasis comprising administering to a subject in need thereof a therapeutically effective amount of an antibody against interferon γ.

This application is a non-provisional U.S. Application claiming the benefit of priority of a provisional application 60/383,310, filed May 22, 2002, and a provisional application resulting from the conversion from a non-provisional application No. 10/150,742, filed May 17, 2002, each of which is herein incorporated by reference in its entirety.

### FIELD OF THE INVENTION

This invention relates to the treatment of autoimmune diseases. In particular, it concerns methods of treating Crohn's disease and/or psoriasis by administration of anti-interferon γ antibodies.

### BACKGROUND OF THE INVENTION

Psoriasis is one of the most common skin diseases, affecting up to 2 percent of the world population. It is a chronic inflammatory skin disorder clinically characterized by erythematosus, sharply demarcated papules and rounded plaques covered by silvery micaceous scales. The skin lesions of psoriasis are variably pruritic. Traumatized areas often develop lesions of psoriasis (Koebner or isomorphic phenomenon). Additionally, other external factors may exacerbate psoriasis including infections, stress, and medications (e.g., lithium, beta blockers, and anti-malaria medications) (Harrison's Principles of Internal Medicine, 14th Edition, pp. 300 (1998)).

Treatment of psoriasis depends on the type, location and extent of disease. Most patients with localized plaque-type psoriasis can be managed with midpotency topical glucocorticoids. However the long-term use of these agents is often accompanied by the loss of effectiveness. Ultraviolet light is an effective therapy for patients with widespread psoriasis. But long-term use of UV light may be associated with an increased incidence of squamous cell cancer of the skin (Harrison's Principles of Internal Medicine, 14^{th} Edition, pp. 300 (1998)).

Various other agents may be used to treat widespread psoriatic disease. For instance, methotrexate is an effective agent, especially in patients with associated psoriatic arthritis. However, due to liver toxicity, its long-term use is limited for patients with widespread disease who are not responsive to less aggressive modalities. Similarly, the synthetic retinoid, etretinate, has been shown to be effective in some patients with severe psoriasis, but it is a potent teratogen with an extremely long tissue half-life, thus precluding its use in women with childbearing potential (Harrison's Principles of Internal Medicine, 14^{th} Edition). Cyclosporine is also effective but its use is limited by its toxicity.

Crohn's disease is an inflammation primarily of the small intestine that can also affect the esophagus, stomach, colon, and other organs and tissues. The prevalence of the disease in the United States is estimated at approximately 100 cases per 100,000 individuals. Symptoms of Crohn's disease include fever, abdominal pain, diarrhea, weight loss, and generalized fatigability. Recurrence is common and unpredictable, and severely affects the patient's quality of life. Current drug treatments for Crohn's disease include anti-inflammatory and immunosuppressive agents including antagonists of TNF-α such as Remicaide®. However, the response to these agents frequently decreases over time and the disease often becomes chronic, leading in many cases to repeated surgical intervention (Harrison's Principles of Internal Medicine, 14^{th} Edition, pp. 1643 (1998)).

In view of the deficiency of the existing treatment approaches, it is of great significance to pursue new methods of treatment for both Crohn's disease and psoriasis. Although the exact cause of Crohn's disease or psoriasis is not yet known, both diseases appear, at least in part, to be autoimmune diseases.

Interferon-γ is a cytokine that functions in the regulation of the immune system and may lead to a type of immune response typical of autoimmune diseases. Increased production of interferon-γ has been demonstrated in T-cells derived from the lamina propria of Crohn's disease patients' lesion, but not in the gut tissue with ulcerative colitis, and antibodies against interferon-γ have been demonstrated to be effective in an animal model of Crohn's disease (Powrie, et. al., Immunity 1: 553-562 (1994)). Serum levels of interferon γ are significantly higher in psoriatic patients compared to normal subjects (Chodorowska G. J. Eur. Acad. Dermatol. Venereol. 10: 147-151 (1998); Gomi T. et al., Arch Dermatol. 127: 827-830 (1991)). Psoriatic skin contains elevated number of interferon γ producing T-cells (Szabo S. K., et. al., J. Invest. Dermatol. 111(6): 1072-1078 (1998)). Elevated interferon γ levels appear to correlate with disease severity (Gomi T., et. al.). An anti-interferon γ antibody has been shown to alleviate some symptoms in an animal model of psoriasis (see Ehrhardt et al., U.S. Pat. No. 6,410,824, This and other U.S. patents/patent applications are herein incorporated by references in their entirety). U.S. Pat. No. 6,036,956 discloses a method for treating systemic lupus erythematosis by administering polyclonal or monoclonal antibodies specific for interferon γ. U.S. Pat. No. 6,333,032 and U.S. Pat. No. 6,329,511 discuss the possible clinical application of interferon γ for treating autoimmune diseases.

Although the above studies may suggest a possible correlation between interferon γ and autoimmune diseases, clinical studies so far have not been conducted to establish a treatment regimen for either Crohn's disease or psoriasis by using anti-interferon γ antibodies. The present invention discloses a number of clinical studies for the treatment of psoriasis and/or Crohn's disease. Unique and clinically effective treatment regimens are provided in the present invention.

### SUMMARY OF THE INVENTION

The present invention provides a method for the treatment of diseases of the immune system, such as autoimmune diseases.

The present invention provides a method for treating Crohn's disease in a subject in need of such a treatment, comprising administering to the subject a therapeutically effective amount of an antibody against interferon γ. Said treatment reduces the symptoms of the disease, as measured, e.g., by the Crohn's Disease Activity Index (CDAI) score (see Table 1) of said subject. Preferably, the antibody is neutralizing, i.e., neutralizes one or more or all biological activities of interferon γ. Preferably, the antibody is a humanized or human antibody. Most preferably, the antibody is HuZAF (see U.S. Pat. No. 6,329,511) or an antibody that recognizes the same epitope as HuZAF.

The present invention also provides a method for treating psoriasis in a subject in need of such a treatment, comprising administering to the subject a therapeutically effective amount of an antibody against interferon γ. The treatment reduces the symptoms of the disease, as measured, e.g., by the Psoriasis Area and Severity Index (PASI) (see Table 2) score of said subject. Preferably, the antibody is neutralizing, i.e., neutralizes one or more or all biological activities of interferon γ. Preferably, the antibody is a humanized or human antibody. Most preferably, the antibody is HuZAF or an antibody that recognizes the same epitope as HuZAF.

### DETAILED DESCRIPTION OF THE PREFERRED EMBODIMENTS

Definitions:

As used herein, the term “antibody” or “immunoglobulin” is intended to encompass both polyclonal and monoclonal antibodies. The preferred antibody is a monoclonal antibody reactive with interferon γ. The term “antibody” is also intended to encompass mixtures of more than one antibody reactive with interferon γ (e.g., a cocktail of different types of monoclonal antibodies reactive with interferon γ). The term “antibody” is further intended to encompass whole antibodies, biologically functional fragments thereof, single-chain antibodies, and chimeric antibodies comprising portions from more than one species, bifunctional antibodies and antibody conjugates and humanized or human antibodies. Biologically functional antibody fragments, which can also be used, are those peptide fragments derived from an antibody that are sufficient for binding to interferon γ.

By “a therapeutically effective” amount of a drug or pharmacologically active agent or pharmaceutical formulation is meant a sufficient amount of the drug, agent or formulation to provide the desired effect.

A “subject,” “individual” or “patient” is used interchangeably herein, which refers to a vertebrate, preferably a mammal, more preferably a human.

The term “epitope” includes any protein determinant capable of specific binding to an immunoglobulin or an antibody. Epitopic determinants usually consist of active surface groupings of molecules such as amino acids or sugar side chains and usually have specific three-dimensional structural characteristics, as well as specific charge characteristics. Two antibodies are said to bind to the same epitope of a protein if amino acid mutations in the protein that reduce or eliminate binding of one antibody also reduce or eliminate binding of the other antibody, and/or if the antibodies compete for binding to the protein, i.e., binding of one antibody to the protein reduces or eliminates binding of the other antibody.

The term “derived from” means “obtained from” or “produced by”.

The term “genetically altered antibodies” means antibodies wherein the amino acid sequence has been varied from that of a native antibody. Because of the relevance of recombinant DNA techniques to this invention, one need not be confined to the sequences of amino acids found in natural antibodies; antibodies can be redesigned to obtain desired characteristics. The possible variations are many and range from the changing of just one or a few amino acids to the complete redesign of, for example, the variable or constant region. Changes in the constant region will, in general, be made in order to improve or alter characteristics, such as complement fixation, interaction with membranes and other effector functions. Changes in the variable region will be made in order to improve the antigen binding characteristics.

The term “humanized antibody” or “humanized immunoglobulin” refers to an immunoglobulin comprising a human framework, at least one and preferably all complementarity determining regions (CDRs) from a non-human antibody, and in which any constant region present is substantially identical to a human immunoglobulin constant region, i.e., at least about 85-90%, preferably at least 95% identical. Hence, all parts of a humanized immunoglobulin, except possibly the CDRs, are substantially identical to corresponding parts of one or more native human immunoglobulin sequences. See, e.g. Winter et al., U.S. Pat. No. 5,225,539; Queen et al., U.S. Pat. Nos. 5,530,101, 5,585,089, and 6,180,370 (each of which is incorporated by reference in its entirety).

The term chimeric antibody refers to an antibody in which the constant region comes from an antibody of one species (typically human) and the variable region comes from an antibody of another species (typically rodent).

The present invention provides a method of preventing or treating diseases of the immune system, particularly autoimmune diseases, by using anti-interferon γ antibodies. The autoimmune diseases include, but are not limited to, Addison's disease, autoimmune diseases of the ear, autoimmune diseases of the eye such as uveitis, autoimmune hepatitis, Crohn's disease, diabetes (Type I), epididymitis, glomerulonephritis, Graves' disease, Guillain-Barre syndrome, Hashimoto's disease, hemolytic anemia, systemic lupus erythematosus, multiple sclerosis, myasthenia gravis, pemphigus vulgaris, psoriasis, rheumatoid arthritis, sarcoidosis, scleroderma, Sjogren's syndrome, spondyloarthropathies, thyroiditis, ulcerative colitis and vasculitis.

The present invention provides a method for the treatment of Crohn's disease and other inflammatory bowel diseases such as ulcerative colitis in a subject in need thereof, comprising administering to the subject a therapeutically effective amount of an antibody recognizing interferon γ. The treatment decreases the severity of Crohn's disease as manifested, e.g., by a reduction of the CDAI score of said subject. The CDAI score is described in Table 1. Preferably, the CDAI score is reduced by at least 70 points (“response”) through treatment disclosed by the present invention. More preferably, the Crohn's treatment of the present invention reduces the CDAI of the Crohn's disease patient by at least 100 points (“enhanced response”). Even more preferably, the CDAI score of the Crohn's sufferer is reduced to an absolute score of 150 points or less (“remission”). When applied to a population of Crohn's disease patients, treatment with the anti-interferon γ antibody will lead to responses, enhanced responses or remissions in at least 20% or 30%, but preferably 40% or 50% or even 60%, more preferably 70% or 80% and most preferably 90% or more of the patients. Preferably this effect should be demonstrated in a clinical trial, for example a phase II or phase III clinical trial, and the increase in responses, enhanced responses or remissions relative to the control group (not treated with the anti-interferon γ antibody) should be statistically significant. The CDAI score can be measured at about 28 days or 1, 2, 3, 4 or 6 months after beginning or end of treatment, or at some other convenient time.

The present invention also provides for a method for the treatment of psoriasis in a subject in need of such a treatment, comprising administering to the subject a therapeutically effective amount of an antibody against interferon γ. The treatment should cause a reduction of PSAI score of said subject. The PASI score is well known to those of skill in treating skin diseases and defined in Table 2. Treatment with the antibody should cause a reduction of at least 50% (PASI50) or 75% (PASI75) in the PASI score or even complete or near-complete clearance of the psoriatic lesions. When applied to a population of psoriasis patients, treatment with the anti-interferon γ antibody will lead to PASI50, PASI75 or clearance in at least 20% or 30%, but preferably 40% or 50% or even 60%, more preferably 70% or 80% and most preferably 90% or more of the patients. Preferably this effect should be demonstrated in a clinical trial, for example a phase II or phase III clinical trial, and the increase in PASI50, PASI75 or clearance relative to the control group (not treated with the anti-interferon γ antibody) should be statistically significant. The PASI score can be measured at about 28 days or 1, 2, 3, 4 or 6 months after beginning or end of treatment, or at some other convenient time.

Anti-interferon γ antibodies for use in the present invention include antibodies that bind to any epitope of interferon γ. They include natural and genetically altered anti-interferon γ antibodies of all species origins. Non-limiting exemplary natural anti-interferon γ antibodies include anti-interferon γ antibodies derived from human, chicken, goats, and rodents (e.g., rats, mice, hamsters and rabbits), including transgenic rodents genetically engineered to produce human antibodies (see, e.g., Lonberg et al., WO93/12227; U.S. Pat. No. 5,545,806; and Kucherlapati, et al., WO91/10741; U.S. Pat. No. 6,150,584, which are herein incorporated by reference in their entirety).

Antibodies useful in the present invention also may be made using phage display methods (see, e.g., Dower et al., WO91/17271 and McCafferty et al., WO92/01047, U.S. Pat. No. 5,969,108, which are herein incorporated by reference in their entirety). For use in human patients, the antibodies must bind to human interferon γ. The antibodies should have binding affinity for interferon γ of at least 10⁷ M⁻¹ but preferably at least 10⁸ M⁻¹, more preferably at least 10⁸ M⁻¹, most preferably 10⁹ M⁻¹ and ideally 10¹⁰ M⁻¹ or higher. The affinity of the antibodies may be increased by in vitro mutagenesis using phage display or other methods (see, e.g., Co, et al., U.S. Pat. No. 5,714,350, which is herein incorporated by reference in its entirety). Preferably, the antibodies will be neutralizing, that is, they will neutralize at least one but most preferably all biological properties of interferon γ, for example stimulation of MHC expression on suitable cells, activation of macrophages, stimulation of Th1 cell development, and anti-viral activity. The antibodies will generally inhibit or block binding of interferon γ of to its cellular receptor. The polyclonal forms of these antibodies can be produced in non-human host animals by immunization with human interferon γ. The monoclonal antibodies can be produced by immunization and hybridoma methodology. The hybridoma methodology and immunization procedure are well known in the art.

Recombinant DNA techniques can be used to produce recombinant anti-interferon γ antibodies, which are also included in the present invention. The amino acid sequence of such recombinant antibodies can be identical to the sequences of amino acids found in natural antibodies. Alternatively, it can be genetically altered so that the amino acid sequence has been varied from that of a native antibody. Recombinant anti-interferon γ antibodies include antibodies produced by any expression systems including both prokaryotic and eukaryotic expression systems. Exemplary prokaryotic systems are bacterial systems that are typically capable of expressing exogenously introduced sequences at large quantity. Illustrative eukaryotic expression systems include fungal expression systems, viral expression systems involving eukaryotic cells such as insect cells, plant cells and especially mammalian cells (such as CHO cells and myeloma cells such as NS0 and SP2/0) which are well-known to those of skill in the art. The antibodies may also be produced by chemical synthesis. However they are produced, the antibodies will be purified by art-known methods such as filtration, chromatography (e.g., affinity chromatography such as by protein A, cation exchange chromatography, anion exchange chromatography, and gel filtration). The minimum acceptable purity of the antibody for use in pharmaceutical formulations will be 90%, with 95% preferred, 98% more preferred and 99% or higher most preferred.

Preferably, the genetically altered anti-interferon γ antibodies used in the present invention include humanized antibodies that bind to and neutralize interferon γ. Examples of these humanized antibodies are disclosed in the U.S. Pat. No. 6,329,511, which is hereby incorporated by reference in its entirety. An exemplary, preferred humanized anti-interferon γ antibody is HuZAF, comprising a mature light chain variable region, whose amino acid sequence is amino acids 21 through 128 of SEQ ID NO 1, and a mature heavy chain variable region, whose amino acid sequence is amino acids 20 through 136 of SEQ ID NO 2. Other preferred antibodies include those that bind to the same epitope of interferon γ as HuZAF, especially other humanized forms of the AF2 antibody described in U.S. Pat. No. 6,329,511. The antibody may be of any of the recognized isotypes, but the four IgG isotypes are preferred, with IgG1 especially preferred. Antibodies with constant regions mutated to have reduced effector function, for example the IgG2m3 and other IgG2 mutants described in U.S. Pat. No. 5,834,597 (which is incorporated by reference in its entirety), are another preferred choice.

The genetically altered anti-interferon γ antibodies also include chimeric antibodies that bind to and neutralize interferon γ. Preferably, the chimeric antibodies comprise a variable region derived from a mouse or rat and a constant region derived from a human so that the chimeric antibody has a longer half-life and is less immunogenic when administered to a human subject. The method of making chimeric antibodies is known in the art.

The fragments of the above-described anti-interferon γ antibodies, which retain the binding specificity to interferon γ, are also included in the present invention. Examples include, but are not limited to, the heavy chains, the light chains, and the variable regions as well as Fab and (Fab′)₂ of the antibodies described herein.

The genetically altered antibodies also include modified anti-interferon γ antibodies that are functionally equivalent to above antibodies and antibody fragments. Modified antibodies providing improved stability and/or therapeutic efficacy are preferred. Examples of modified antibodies include those with conservative substitutions of amino acid residues, and one or more deletions or additions of amino acids which do not significantly deleteriously alter the antigen binding utility. Substitutions can range from changing or modifying one or more amino acid residues to complete redesign of a region as long as the therapeutic utility is maintained. Antibodies of this invention can be can be modified post-translationally (e.g., acetylation, and phosphorylation) or can be modified synthetically (e.g., the attachment of a labeling group). Fragments of these modified antibodies that retain the binding specificity can also be used.

The present invention provides a pharmaceutical formulation comprising the antibodies described herein. Pharmaceutical formulations of antibodies are prepared for storage by mixing the antibodies having the desired degree of purity with optional physiologically acceptable carriers, excipients, or stabilizers, in the form of lyophilized or aqueous solutions. Acceptable carriers, excipients or stabilizers are nontoxic to recipients at the dosages and concentrations employed, and include buffers such as phosphate, citrate, and other organic acids; antioxidants, preservatives, low molecular weight polypeptides, proteins, hydrophilic polymers, amino acids, carbohydrates, chelating agents, sugar, and other standard ingredients known to people skilled in the art (Remington's Pharmaceutical Science 16^{th} edition, Osol, A. Ed. 1980).

The formulation herein may also contain more than one active compound as necessary for the particular indication being treated, preferably those with complementary activities that do not adversely affect each other. Such molecules are suitably present in combination in amounts that are effective for the purpose intended.

Active ingredients of the above pharmaceutical formulation may also be entrapped in microcapsules, in colloidal drug delivery systems (for example, liposome, albumin microspheres, microemulsions, nano-particles and nanocapsules), in macroemulsions, or in sustained-release preparation. Such techniques are known to people skilled in the art (Remington's Pharmaceutical Sciences).

The formulation to be used for in vivo administration is usually stored at 2 to 8° C. The formulations often contain no preservatives and should be used within 4, 12 or 24 hours of withdrawal from the vial and dilution into saline. The formulation is preferably administered intravenously or subcutaneously with or without filtration. Preferably, humanized anti-interferon γ antibody, HuZAF is stored in a single-use glass vial containing 1.0 mL of HuZAF at a concentration of 50 mg/mL in an isotonic buffer of histidine, glycine, and Polysorbate 80, at pH 6.0. However, concentrations from 1 to 10 mg/mL (e.g., 1, 2, 5 or 10), 20 to 50 mg/mL (e.g., 20, 30, 40 or 50) or 60 to 100 mg/mL (e.g., 60, 70, 80, 90 or 100) are also possible.

The antibodies prepared in a pharmaceutical formulation can be administered by any suitable route including oral, rectal, nasal, topical (including transdermal, aerosol, buccal and sublingual), parental (including subcutaneous, intramuscular, intravenous and intradermal) or by inhalation therapy. It will also be appreciated that the preferred route may vary with the condition and age of the recipient.

Preferably, the pharmaceutical formulation is delivered via intravenous infusion, for example over 30 or 60 minutes, such that a therapeutically effective amount of said composition is delivered via systemic absorption and circulation. Alternatively, the pharmaceutical formulation is delivered via subcutaneous injection, such that a therapeutically effective amount of said composition is delivered via systemic absorption and circulation. The formulation may also be delivered as an intravenous bolus.

A therapeutically effective amount of above formulations depends on the severity of the Crohn's disease or psoriasis, the patient's clinical history and response, and the discretion of the attending physician. The composition is suitably administered to the patient at one time or over a series of treatments. The initial candidate dosage may be administered to a patient. The proper dosage and treatment regime can be established by monitoring the progress of therapy using conventional techniques known to the people skilled of the art.

The amount of active ingredients that may be combined with the carrier materials to produce a single dosage form will vary depending upon the subject treated and the particular mode of administration. It will be understood, however, that the specific dose level for any particular patient will depend upon a variety of factors, including the activity of the specific composition employed, the age, body weight, general health, sex, diet, time of administration, route of administration, rate of excretion, drug combination and the severity of the particular disease undergoing therapy, and can be determined by those skilled in the art.

In particular, an exemplary effective dose for the treatment of Crohn's disease or psoriasis ranges between about 0.01 mg/kg to about 100 mg/kg, preferably between about 0.1 mg/kg to about 10 mg/kg, and more preferably about 0.5 mg/kg to about 5 mg/kg. The dose is expected to be well-tolerated based on the pre-clinical trial studies in animals and humans. Preferred dose levels include 0.1 mg/kg, 0.5 mg/kg, 1 mg/kg, 2 mg/kg, 4 mg/kg, 5 mg/kg and 10 mg/kg. An initial higher “loading dose” of the antibody may be followed by lower maintenance doses, for example 2, 4, 5 or 10 mg/kg may be followed by 0.1, 0.5, 1 or 2 mg/kg. The loading dose may be administered intravenously followed by maintenance doses administered subcutaneously. Fixed unit doses may also be administered, e.g., 50, 100 or 200 mg.

Depending on the progress in treatment and the physical conditions of the patients, the regimen of the treatment of Crohn's disease and psoriasis can vary significantly. For both Crohn's disease and psoriasis, a patient is administered at least a single dose of said pharmaceutical formulation. Additional doses can be administered for multiple times (for example, once, twice, three times, four times, five times, 6-10 times or more), e.g., daily, two or three times a week, biweekly, or monthly, every 6 weeks, or every 2 or 3 months. When multiple doses are administered, the period of treatment may be, e.g., 1, 2, 4 or 6 weeks, or 2, 3, 4, 6 or 12 months or indefinitely. The patient may receive 2, 3, 4 or more courses of treatment if the disease relapses.

As a preferred treatment regimen for Crohn's disease, a single dose of anti-interferon γ antibody is administered to a patient by intravenous infusion. After 4 weeks, the CDAI score of the patient receiving the treatment is evaluated. If a reduction of more than 70 points is observed, the patient will receive a dose that is 50% of the initial single dose in a regimen of one dose every 4 weeks. The 50% dose is needed to avoid drug accumulation due to the 2 to 3 week half-life of HuZAF. The initial dose may be 0.1, 1, 4 or 10 mg/kg.

Alternatively, the combination of intravenous infusion and subcutaneous injection of the antibodies are used for the treatment of Crohn's disease. A patient is both subcutaneously injected and intravenously infused (preferably, for over 30 minutes) with the antibodies. Preferably, a patient receives antibodies as an IV infusion on Day 1. Beginning on Day 29, the patient receives one SC dose every 4 weeks for 3 doses (Day 29, Day 57, and Day 85). CDAI evaluation is performed at Day 28 and each treatment day and Day 113. Patients are monitored to determine if additional SC administration is needed to maintain the response. Examples of the treatment regimen include, but are not limited to, (1) about 1.0 mg/kg IV followed by about 0.1 mg/kg SC; (2) about 1.0 mg/kg IV followed by about 1.0 mg/kg SC; (3) about 4.0 mg/kg IV followed by about 0.1 mg/kg SC; (4) about 4.0 mg/kg IV followed by about 1.0 mg/kg SC.

For efficacy of the treatment of Crohn's disease, patients are scored for CDAI, and by endoscopy for Crohn's Disease Endoscopic Index of Severity (CDEIS; see Table 3). Circulating levels of C-reactive protein (CRP), interleukin-6 (IL-6), interferon-γ-inducible protein-10 (IP-10), and interferon-γ (IFN-γ) are also determined. Biopsies are taken from active lesions, where permitted. Biopsy materials are evaluated for inflammatory activity.

For psoriasis, skin biopsies are performed on selected patients to evaluate the mechanism of action of the anti-interferon γ antibodies. The Psoriasis Area Severity Index (PASI) score is collected to assess any changes in disease activity over time.

The following examples are offered by way of illustration and not by way of limitation. The disclosure of all citations in the specification is expressly incorporated herein by reference for all purposes.

### EXAMPLES

### Example 1

This example describes the pre-clinical studies of anti-interferon γ antibodies in animal models (chimpanzees).

Description of HuZAF

HuZAF, the arbitrary name given to a particular humanized anti-INF-γ antibody developed by the assignee of the present invention, is the humanized form of a murine anti-human IFN-γ antibody (AF2) directed against recombinant human IFN-γ. HuZAF prevents INF-γ from binding to its cellular receptors, thereby neutralizing IFN-γ-mediated activities, including induction of MHC class II molecule expression, viral protection, and inhibition of proliferation of certain cells. The isotype of the HuZAF heavy chain is human IgG1; the light chain is human kappa.

Single-Dose Study in Chimpanzees

Because HuZAF only cross-reacts with the INF-γ of great apes, the safety and PK studies have been restricted to chimpanzees. A single-dose, dose-ranging study in healthy chimpanzees demonstrated no adverse clinical effects after a single intravenous infusion of 2 mg/kg or 20 mg/kg (n=4 at each dose). The population typical values for the distribution half-life and for the elimination half-life were 21.1 hours and 349 hours (14.5 days) respectively. Three of the 4 animals treated with 20 mg/kg showed an unexplained decrease in the ratio of neutrophils to lymphocytes during the 42-day study period. It was noted that the high-dose cohort animals were much older than the controls. One animal in the high-dose around exhibited an increase in the percentage and absolute number of cosinophils. In the immunogenicity assay, positive responses were detected in 2 of 4 chimpanzees administered 2.0 mg/kg HuZAF (response from one of the animals was detected in a predose sample), and transient positive responses were detected in 2 of 4 chimpanzees administered 20 mg/kg of HuZAF. The PK profile of HuZAF in these animals was not affected by the positive responses, so they did not appear to be true anti-HuZAF responses.

### Example 2

This example describes the overall plan of the clinical studies of anti-interferon γ antibodies. The “ZAF xxx” nomenclature is an arbitrary numbering system devised by the assignee of the present invention to designate its various HuZAF antibody clinical studies.

Healthy Volunteers

ZAF 701: Phase I open-label, single IV dose, dose escalation study

ZAF 704: Phase I blinded, placebo-controlled, single SC dose, dose escalation study

Crohn's disease

ZAF 702: Phase I/II double-blind, placebo-controlled, single and multiple IV dose, dose escalation study in moderate to severe CD patients

ZAF 707: Phase II, double-blind (except to the site pharmacist), randomized, placebo-controlled study of HuZAF administered intravenously as a loading dose followed by multiple SC maintenance doses.

ZAF 708: Phase II, randomized, double-blind, placebo-controlled study to determine the safety and efficacy of HuZAF in patients with moderate to severe Crohn's disease

Psoriasis

ZAF 705: Phase I double-blind, placebo-controlled, single IV dose, dose escalation study in patients with plaque psoriasis

ZAF 706: Phase I/II double-blind, placebo-controlled, multiple SC doses, dose escalation study in patients with plaque psoriasis

### Example 3

This example describes the clinical studies in healthy volunteers (ZAF-701) and ZAF-704). ZAF-701 was a single-dose, phase I study designed to assess the safety, pharmacokinetics (PK), and pharmacodynamic (PD) effect of HuZAF administered as an intravenous infusion to 22 healthy volunteers (18 male and 4 female). Six dose levels of HuZAF were tested: 0.01, 0.03, 0.1, 0.3, 1, and 4 mg/kg. Each dose cohort included 4 subjects, with the exception of the 0.03 mg/kg cohort, which included 2 subjects.

There were no deaths or serious adverse events reported in this study. No adverse events deemed probably or definitively related to HuZAF were reported. Headache (7 patients), somnolence (5), and local skin reactions (redness, swelling) at the injection sites (5) were the most frequently cited AEs. Headache was reported at the 0.01, 0.1, 0.3, 1.0, 4.0 mg/kg dose levels; somnolence at the 0.03, 0.1, and 1.0 mg/kg dose levels; and skin reactions at the 0.1, 1.0, and 4.0 mg/kg dose levels.

Pharmacokinetic analyses showed that maximum concentrations of HuZAF observed within the first day of dosing averaged 0.16 to 78 μg/mL and increased proportionally with each dose administered (0.01 to 4 mg/kg). HuZAF exhibited biphasic elimination with a low serum clearance (0.14 mL/hr/kg). The elimination half-life was long and dose-dependent, ranging from 330 to 593 hours (approximately 13.8 to 24.7 days), with the longer half-lives associated with the smaller doses.

Human anti-humanized antibody (HAHA) responses and total INF-γ concentrations were measured. No immunogenicity responses were detected in any of the samples tested. HuZAF administered at 0.3 mg/kg or higher resulted in a slight increase of total serum IFN-γ at later time points (8 hours after dosing). However, the maximum total IFN-γ serum concentration observed in all subjects was less than 66 pg/mL at all time-points.

The PD effect of HuZAF was measured using an antigen challenge to mumps and Candida antigens. Subjects were pre-screened for delayed-type hypersensitivity (DTH) responses to these antigens prior to dosing with HuZAF. At 48 hours after dosing, reduced DTH responses were seen in most of the tested subjects, especially if their screening DTH responses had been vigorous. In subjects who received doses of 0.03 mg/kg or higher, the DTH responses were less than half the size of those observed at screening in 23 of 29 (79%) evaluable tests. The median post-treatment lesion size was 9% of the screening size, which represents a 91% reduction in lesion size. This result indicates the effectiveness of anti-interferon γ antibodies in a model of inflammation, especially of the skin, such as psoriasis.

### Example 4

This example describes a clinical study of phase I/II, double-blind, placebo-controlled, single- and multiple-dose, dose escalation study of a humanized anti-interferon-γ antibody (HuZAF) in patients with moderate to severe Crohn's Disease.

| | |
|---|---|
| Protocol Number: | ZAF-702 |
| Phase: | I/II |
| Study Drug: | HuZAF (in an isotonic buffer composed of L-histidine, |
| | glycine, and Tween 80, at pH 6.0) |
| Comparative | Placebo: buffer only, in the same vial configuration as |
| Drug: | the active drug |
| Doses: | Placebo (zero dose) or HuZAF at either 0.1, 1, |
| | or 4 mg/kg |
| Dosage Form and | Single-use glass vial containing 5.0 mL of a solution |
| Strength: | of HuZAF formulated at a concentration of 10 mg/mL |
| Route: | Intravenous infusion over 60 minutes |
| Diluent: | If the volume to be administered is <50 mL, dilute the |
| | drug to 50 mL in 0.9% saline. If the volume is 50 mL, |
| | infuse without dilution. |
| Storage and | Store under controlled, refrigerated conditions at 2 to |
| Administration: | 8° C. The formulation contains no preservative and is |
| | used within 12 hours of withdrawal from the vial and |
| | dilution (if any) into saline. The formulation must be |
| | administered intravenously without filtration. |

Synopsis:

Protocol ZAF-702 was a Phase I/II, double-blind, randomized, placebo-controlled, single- and multiple dose, dose-escalation study that was conducted at up to 10 investigational sites in 2 stages. In Stage A, up to 48 patients with moderate to severe Crohn's disease between the ages of 18 and 65 received a single dose of HuZAF by intravenous infusion at one of 3 dose levels (0.1, 1, 4 mg/kg) or placebo. Up to 8 additional patients (maximum of 56 patients) may receive HuZAF/placebo if treatment-related adverse events (AE) of clinical concern caused dose escalation to cease at a given dose level. The patients were randomized to receive HuZAF or placebo in a 3:1 ratio. Patients must have had a diagnosis of Crohn's disease for at least 6 months and have received prior treatment for their CD (excludes treatment-naive patients), with a Crohn's Disease Activity Index (CDAI) score of ≧250 and ≧450.

If a ≧70-point reduction of the CDAI score is seen at 4 weeks in a patient who received HuZAF, and if the patient had no treatment-related AEs of clinical concern attributable to HuZAF, the patient would enter Stage B of the study. In Stage B, the patients were re-randomized to receive either 3 doses of HuZAF at a level that is 50% of the single dose received in Stage A of the study, or 3 doses of placebo, in a regimen of one dose every 4 weeks. The 50% dose was needed to avoid drug accumulation due to the anticipated 2- to 3-week half-life of HuZAF. The patients were randomized to receive HuZAF or placebo in a 3:1 ratio. If a patient returned to a pre-Stage A baseline, or worse, CDAI score during Stage B of the study, the patient would leave the study and receive appropriate rescue medication. Patients who received placebo in Stage A would have the option of receiving HuZAF on an open-label basis in a separate study (Protocol ZAF-703).

General Results of ZAF-702 Clinical Study

As to Stage A, a Phase I/II, double-blind, placebo-controlled, dose escalation study was conducted to measure the safety and efficacy of a single IV infusion of HuZAF in patients with moderate to severe active Crohn's disease (CD). Forty-five patients (of whom 42 were evaluable) with CD who had CDAI scores of more than 250 and less than 450 were randomized to receive one of 3 dose levels of HuZAF (0.1, 1.0, or 4.0 mg/kg) or a single dose of placebo. Study endpoints at 4 weeks postdosing were (1) clinical response (decrease of CDAI score more than or equal to 70 points from baseline), (2) remission (CDAI score of less or equal to 150), (3) enhanced response (decrease of CDAI score more than or equal to 100 points), and (4) safety. The results showed that demographics and baseline disease characteristics were comparable for the 4 groups. Adverse events were distributed among the 4 groups; the 3 reported SAEs were considered to be related to disease. Increased doses of HuZAF correlated with higher rates of clinical response and greater numbers of remissions. Likewise, the percent of patients with enhanced response was higher as dose levels increased (Table 4). In conclusion, the results indicate that HuZAF administered as a single IV dose to patients with moderate to severe active CD is well tolerated with a good safety profile. Increasing doses of HuZAF yielded higher clinical response rates and greater numbers of remissions, indicating that HuZAF is effective for the treatment of Crohn's disease.

At Stage B, patients who responded in Stage A were re-randomized to receive 50% of stage A dose or placebo every 4 weeks for 3 doses. At study day 113, most patients retained their response or remission, whether they received additional doses of HuZAF or placebo, indicating that responses and remissions induced by HuZAF are generally long-lasting.

As shown in Table 4, at the lowest dose of HuZAF (0.1 mg/kg, n=6), 50% of the patients responded and none achieved a remission. At the dose of 1.0 mg/kg (n=12), 67% of the patients responded and 25% achieved a remission, and at the highest dose tested, 4.0 mg/kg (n=14), 71% of the patients responded and 50% achieved a remission. In the placebo group (n=10), 60% of the patients responded and 40% achieved a remission.

TABLE 4
Number (Percent) of Patients with Clinical Response and
Remission at Day 29

| | Number of | | Enhanced | |
|---|---|---|---|---|
| | Patients | Responses | Responses | Remissions |
| Placebo | 10 | 6 (60%) | 6 (60%) | 4 (40%) |
| 0.1 mg/kg | 6 | 3 (50%) | 1 (17%) | 0 (0%) |
| 1 mg/kg | 12 | 8 (67%) | 5 (42%) | 3 (25%) |
| 4 mg/kg | 14 | 10 (71%) | 8 (57%) | 7 (50%) |

### Example 5

This example describes a Phase I, double-blind, placebo-controlled, single-dose, dose escalation study to evaluate the safety, tolerability, and pharmacokinetics of HuZAF, a humanized anti-interferon-gamma antibody, in patients with plaque psoriasis.

| | |
|---|---|
| Protocol Number: | ZAF-705 |
| Phase: | Phase I |
| Countries: | United States |
| | (Canada) |
| Indication: | Plaque psoriasis |
| Study Drug: | HuZAF |
| Comparative Drug: | Placebo, isotonic buffer composed of histidine, |
| | glycine, and Polysorbate 80, at pH 6.0 |
| Dose: | 0 (placebo), 0.1, 1.0, 3.0, 10.0 mg/kg |
| Dosage Form and | Single-use glass vials containing 1.0 mL of HuZAF |
| Strength: | at a concentration of 50 mg/mL formulated in an |
| | isotonic buffer of 20 mM histidine, 275 mM |
| | glycine, and 0.01% Polysorbate 80 at pH 6.0 |
| Route: | Intravenous infusion over 30 minutes |
| Diluent: | Saline 0.9% |
| Storage, Filtration, | Store under controlled, refrigerated conditions at |
| and Administration: | 2 to 8° C. The formulation contains no preservative |
| | and must be used within 12 hours of withdrawal |
| | from the vial. The formulation must be administered |
| | intravenously without filtration. |

Synopsis:

ZAF-705 was a Phase I, double-blind (but not to the sponsor and the site pharmacist), randomized, single dose, dose escalation trial of HuZAF that is conducted in up to 3 centers in the United States (US) and Canada. Up to 35 patients with plaque psoriasis who are 18 years of age or older received a single dose of 0 (placebo), 0.1, 1.0, 3.0, or 10.0 mg/kg HuZAF by intravenous infusion. The objectives of the study were to evaluate the safety, tolerability, and pharmacokinetics (PK) of a single intravenous dose of HuZAF and the ability of HuZAF to decrease disease activity.

Patients were randomized to receive HuZAF or placebo at the following dose levels: 0.1, 1.0, 3.0 and 10.0 mg/kg HuZAF, which corresponded to 0.002, 0.02, 0.06, and 0.2 mL/kg placebo. Up to 5 additional patients would receive HuZAF/placebo if treatment-related adverse events (AE) of clinical concern caused dose escalation to cease at any given dose level. Patients must not have received systemic therapy for their psoriasis (either approved or investigational) within 30 days prior to study entry. Patients using topical agents must have been on a stable dose for at least 2 weeks prior to study entry. New topical agents would be allowed only if lesions on the scalp, face, or groin worsen. After Day 85, patients receiving placebo who had not experienced ≧50% improvement in their disease status were offered HuZAF at their original assigned dose level.

Treatment and follow-up continued through Day 85 with a long-term follow-up at 6 months.

Randomization, Dose Escalation, and Toxicity Management

Patients were randomized to receive HuZAF/placebo at the following dose levels: 0 (placebo), 0.1, 1.0, 3.0, and 10.0 mg/kg. At each dose level, 5 patients were randomized in a ratio of 4 HuZAF patients to one placebo patient.

Study Measurements and Analyses

Blood samples were collected at specified time periods throughout the study for the determination of pharmacokinetic (PK) and human anti-humanized antibody (HAHA) levels. A complete PK profile was obtained at all dose levels.

Safety was evaluated throughout the treatment and follow-up periods of the study. Clinical status and laboratory values (hematology and chemistry) of all patients were monitored. Adverse events were documented and characterized according to their severity and relationship to HuZAF/placebo. All subjects enrolled in the study were followed for AEs for 3 months and for infections and malignancies for 6 months after receiving HuZAF/placebo.

Skin biopsies were performed on selected patients to evaluate the mechanism of action of HuZAF. The Psoriasis Area Severity Index (PASI) (Table 2) was collected to assess any changes in disease activity over time.

Summary statistics included mean, median, standard deviation, and the minimum and maximum changes in disease state as measured by calculating the change from baseline of the PASI score. Pharmacokinetic results were presented by dose level in tables and graphs. Adverse events and clinical and laboratory assessments were presented in tables.

As shown in Table 5, the PASI score of the tested patients was improved after the treatment with HuZAF in a dose-dependent manner. For instance, at D15, about 25% patients who were treated with HuZAF achieved a more than 50% and 75% reduction of PASI scores. The treatment with 3 mg/kg HuZAF gave rise to a mean reduction of 31.9% in PASI scores while the treatment with 0.1 mg/kg HuZAF gave rise to a mean reduction of 4.3% in PASI scores. One patient had a reduction of PASI scores as high as 76.5%. At D29, about 25% patients who were treated with HuZAF achieved a more than 50% reduction of PASI scores. The treatment with 3 mg/kg HuZAF gave rise to a mean reduction of 21.9% in PASI scores while the treatment with 0.1 mg/kg HuZAF gave rise to a mean reduction of 8.3% in PASI scores.

### Example 6

This example describes a Phase I/II, double-blind, placebo-controlled, multiple-dose, dose escalation study to evaluate the safety, tolerability, pharmacokinetics, and biologic activity of a humanized anti-interferon-γ monoclonal antibody (HuZAF) in patients with plaque psoriasis

| | |
|---|---|
| Protocol Number: | ZAF-706 |
| Phase: | Phase I/II |
| Country/Regulatory Number: | Canada CTA #: 074512 |
| Test Drug: | HuZAF |
| Comparative Drug: | Placebo |
| Dose: | 0.1, 0.5, 1.0 mg/kg |
| Dosage Form and Strength: | Liquid, 50 mg/mL |
| Route of Administration: | Subcutaneous injection |
| Storage, Filtration, and | To be stored at 2 to 8° C. Must be used |
| Administration: | within 3 hours of withdrawal from |
| | the vial. |
| | Six subcutaneous injections every 2 weeks |
| | for the first 15 patients. |
| Dose Interval/Escalation: | Weekly SC injections for 12 doses for the |
| | additional 20 patients enrolled into the |
| | expanded 1 mg/kg dose cohort. |

Synopsis:

ZAF-706 is a Phase I/II, double-blind (not to the sponsor and the site pharmacist), randomized, multiple-dose, dose escalation trial of HuZAF that is conducted in up to 4 centers in Canada. Up to 35 patients with moderate to severe plaque psoriasis who are 18 years of age or older are randomized to receive subcutaneous (SC) injections of placebo or HuZAF (0.1, 0.5, or 1.0 mg/kg). Up to 5 additional patients may receive HuZAF or placebo if treatment-related adverse events (AE) of clinical concern cause dose escalation to cease at any given dose level. The objectives of the study are to evaluate the safety, tolerability, and pharmacokinetics (PK) of HuZAF when administered as multiple SC injections. The ability of HuZAF to decrease disease activity is studied.

Patients must not have received systemic therapy for their psoriasis (either approved or investigational) within 30 days prior to study entry. Patients must not have had prior antibody treatment within 6 months. Patients using topical agents must have been on a stable dose for at least 2 weeks prior to study entry. New topical agents are allowed only if lesions on the scalp, face, or groin worsen. A chest x-ray and tuberculin skin test is performed before the start of the study to rule out latent tuberculosis; patients who test positive are excluded from the study.

Treatment and follow-up continue through Day 131 with a long-term follow-up at 4 and 6 months following administration of the final dose.

Randomization, Dose Escalation, and Toxicity Management

Patients are randomized to receive placebo or HuZAF at the following dose levels: 0.1, 0.5, and 1.0 mg/kg. At each dose level, 5 patients are randomized in a ratio of 4 HuZAF patients to one placebo patient. If 3 out of 4 HuZAF patients at a given dose level experience no study-related AEs of clinical concern within 7 days after receiving the first dose, escalation to the next dose level will occur. If 2 of 4 HuZAF patients experience a study-related AE of clinical concern within 7 days of the first dose, 3 additional patients will be randomized in a 2:1 ratio (HuZAF:placebo) at that same dose level. If either of the 2 HuZAF patients experiences an AE of clinical concern within 7 days of dosing, dose escalation will cease and 5 more patients will be enrolled at the previous dose level to verify the safety profile of that dose level. A total of 6 doses are administered every 2 weeks (Study Days 1, 15, 29, 43, 57, and 71). The 1 mg/kg cohort is expanded to enroll an additional 20 patients. One of 5 patients receives placebo. A total of 12 doses are administered weekly (Study Days 1, 8, 15, 22, 29, 36, 43, 50, 57, 64, 71, and 78).

Study Measurements and Analyses

Blood samples are collected at specified time periods throughout the study for the determination of PK and anti-antibody (anti-Ab) levels. A complete PK profile is obtained at all dose levels.

Safety is evaluated throughout the treatment and follow-up periods of the study. Clinical status and laboratory values (hematology and chemistry) of all patients are monitored with special attention paid to the development of infections. A repeat chest x-ray is performed 2 months after the last dose of the test drug. Adverse events are documented and characterized according to their severity and relationship to HuZAF or placebo. All patients enrolled in the study are followed for AEs for 30 days after receiving the final dose of HuZAF or placebo. Patients are followed for 6 months after receiving the final dose of HuZAF or placebo for infections, including tuberculosis, and malignancies. Skin biopsies are performed on all patients (who consent) to evaluate the mechanism of action of HuZAF. The Psoriasis Area Severity Index (PASI) and Physician's Global Assessment (PGA) scores are collected to assess any changes in disease activity over time.

Summary statistics include mean, median, standard deviation, and the minimum and maximum changes in disease state as measured by calculating the change from baseline of the PASI and PGA scores. Pharmacokinetic results are presented by dose level in tables and graphs. Adverse events and clinical and laboratory assessments are presented in tables.

It is expected that that at least 30% or 50% or 70% of the patients treated in this study will reach PASI50 or PASI75, especially at the 0.5 and/or 1 mg/kg dose levels, and that this percentage will be greater than in the placebo group, indicating that HuZAF is effective for the treatment of psoriasis.

### Example 7

This example describes a Phase II, double-blind, placebo-controlled study to determine the safety and efficacy of a humanized anti-interferon-γ monoclonal antibody (HUZAF) administered to patients with moderate to severe Crohn's disease.

| | |
|---|---|
| Protocol Number: | ZAF-707 |
| Phase: | Phase II |
| Country/Regulatory | US IND #: 10298 |
| Number: | HC IND #: 075932 |
| Test Drug: | HuZAF (in an isotonic buffer composed of histi- |
| | dine, glycine, and Polysorbate 80, at pH 6.0) |
| Comparative Drug: | Placebo (isotonic buffer composed of histidine, |
| | glycine, and Polysorbate 80, at pH 6.0) |
| Doses (IV and SC): | IV: Placebo (zero dose), HuZAF at 1.0 or 4.0 |
| | mg/kg |
| | SC: Placebo (zero dose), HuZAF at 0.1 or |
| | 1.0 mg/kg |
| Dosage Form and | Single-use glass vials containing 1.0 mL of |
| Strength: | HuZAF at a concentration of 50 mg/mL in an |
| | isotonic buffer of histidine, glycine, |
| | and Polysorbate 80, at pH 6.0 |
| Route of | IV: Intravenous infusion over 30 minutes |
| Administration | SC: Subcutaneous injection over ≦5 seconds |
| (IV and SC): | |
| Diluent (IV and SC): | IV: Dilute the test article to 50 mL in 0.9% saline. |
| | SC: Placebo |
| Storage, Filtration, | The formulation contains no preservative, and |
| and Administration: | must be administered without filtration. |
| | IV: The formulation should be used within 12 |
| | hours of withdrawal from the vial and dilution |
| | into saline. |
| | SC: The formulation must be used within 3 hours |
| | of withdrawal from the vial. |

Synopsis:

ZAF-707 is a Phase II, double-blind (except to the site pharmacist), randomized, placebo-controlled study of HuZAF administered intravenously as a loading dose followed by multiple SC maintenance doses that is conducted at up to 25 investigational sites in the US, Canada, and Europe. The objective of the study is to evaluate the safety and efficacy of an IV loading dose of HuZAF for induction of a clinical response, defined as a decrease of ≧100 points in the Crohn's Disease Activity Index (CDAI) score at Day 29. An additional CDAI evaluation is also performed at Day 43 after administration of the IV loading dose and one SC maintenance dose. The ability of HuZAF to maintain the response with subsequent SC administration is also evaluated.

Up to 175 patients with moderate to severe Crohn's disease (CD) between the ages of 18 and 70 are randomized to one of 5 treatment groups: (1) HuZAF 1.0 mg/kg IV followed by 0.1 mg/kg SC; (2) HuZAF 1.0 mg/kg IV followed by 1.0 mg/kg SC; (3) HuZAF 4.0 mg/kg IV followed by 0.1 mg/kg SC; (4) HuZAF 4.0 mg/kg IV followed by 1.0 mg/kg SC; and (5) placebo IV followed by placebo SC. Patients must have had a diagnosis of CD for at least 6 months and have received prior treatment for their CD (excluding treatment-naïve patients and those who received 5-ASA and antibiotics as their only CD treatment), with a CDAI score between 250 and 450 inclusive.

Patients receive HuZAF (1.0 or 4.0 mg/kg) or placebo as an IV infusion on Day 1. Beginning on Day 29, patients receive one SC dose every 4 weeks for 3 doses (Days 29, 57, and 85). Patients who are treatment failures or who require surgery for CD complications at any time will leave the study.

Study Measurements and Analyses

Blood samples are collected at specified time periods throughout the study for the determination of pharmacokinetic (PK) and anti-antibody (anti-Ab) levels. A complete PK profile is obtained at all dose levels for selected patients. For efficacy, patients are scored using the CDAI and Inflammatory Bowel Disease Questionnaire (IBDQ). Circulating levels of C-reactive protein (CRP) are determined. Clinical response is defined as a decrease of ≧100 points in CDAI score from baseline levels without an accompanying increase in dose of concomitant medications or addition of a new medication as therapy for the disease. The primary endpoint is clinical response at Day 29. CDAI scores are evaluated at specified time points throughout the study to assess maintenance of response. IBDQ scores are used to evaluate patients' quality of life.

Safety is evaluated throughout the treatment and follow-up periods of the study. Patients are monitored for the development of antinuclear antibodies and anti-double-stranded DNA antibodies. Clinical status and laboratory values (hematology and chemistry) of all patients are monitored, with special attention paid to the development of infections. Chest radiograms are performed periodically to monitor the potential development of tuberculosis. Adverse events (AE) are documented and characterized according to their severity and relationship to HuZAF or placebo. All patients enrolled in the study are followed for serious adverse events (SAE) and laboratory abnormalities for 90 days after receiving the final dose of HuZAF or placebo. All patients are contacted at 6 months after receiving the final dose of test article to inquire about serious infections, including tuberculosis, and malignancies.

Demographics and baseline characteristics of all patients are summarized collectively and by treatment group. Patients are pooled across sites when partitioned by treatment group and pooled across treatment groups when compared by site. The characteristics of interest include age, sex, ethnicity, baseline CDAI and IBDQ scores, CRP value, disease duration, and smoking status.

Summary statistics include response rates and associated 95% confidence intervals, as well as mean, standard deviation (or error, as appropriate), median, minimum and maximum changes in CDAI and IBDQ scores, and serum levels of CRP from respective baselines to appropriate visits. The magnitude and direction of the changes are compared between treatment groups.

Anti-Ab samples are collected from all patients, and all of the samples will be analyzed. PK samples are collected from odd-numbered patients only; PK samples collected from placebo patients are not analyzed for PK or used for any other purpose.

Assuming a 70% response rate in either of the active IV treatment groups and 35% response in the IV placebo group, the study will have a 91% power to detect a difference of 35 percentage points, α=0.05, with 70 patients for each active IV treatment group and 35 patients for the placebo group for the primary endpoint (total number of patients=175). The percent of patients who achieve remission (absolute CDAI≦150) will also be compared between the placebo and the active treatment groups. The study is not powered to detect statistically significant differences among the five IV-to-SC dosing regimens. Due to anticipated losses of non-responders, fewer than 30 patients per arm are likely.

### Example 8

This example describes a Phase II, double-blind (except to the site pharmacist), randomized, placebo-controlled study to be conducted at up to 25 investigational sites in Europe. Up to 120 patients with moderate to severe CD will receive placebo or HuZAF at a dose of 4 or 10 mg/kg intravenously.

All patients who are enrolled into the study are randomized to receive placebo or HuZAF at 4 or 10 mg/kg in a 1:1:1 ratio (HuZAF:HuZAF:placebo). The randomization is stratified prospectively on disease severity. Specifically, CDAI scores of ≧250 and <350 will be one stratum, while scores of ≧350 and ≦450 are the other stratum. A minimum of 30 patients is randomized into the study to receive a single infusion of placebo or HuZAF at 4 or 10 mg/kg on Study Day 0 (Group 1). CDAI scores are assessed at Study Day 28 and safety data are collected. An independent Data Safety Monitoring Board (DSMB) reviews and evaluates all clinically significant safety information and may make the recommendation to increase the dosing regimen to 2 doses administered 28 days apart. Subsequent patients are randomized to receive one infusion of the test article during the DSMB review. If no major concerns regarding the extent and type of AEs encountered arise and the recommendation to proceed to the next group of patients is made, the remaining patients will be randomized to receive 2 doses of the test article (Group II). An email and a written letter will be sent out to notify each study site to begin enrollment into Group II. Prior to receiving this notification, the sites should continue to enroll patients into Group I.

Patients in Group II receive the first IV infusion of placebo or HuZAF (4 or 10 mg/kg) at Study Day 0. On Study Day 28, after completing all required study assessments, patients receive their second IV infusion of placebo or HuZAF (4 or 10 mg/kg). Patients who are treatment failures (defined below) will leave the study, receive appropriate rescue medications, and be followed for safety.

A treatment failure is defined as a patient whose CDAI score increases ≧100 points from the lowest CDAI score at any time (absolute CDAI score must be greater than 150 points) or increases to a score of ≧450 points.

Study Synopsis

A Phase II, Randomized, Double-Blind, Placebo-Controlled Study to Determine the Safety and Efficacy of HuZAF, a Humanized Anti-Interferon-y Monoclonal Antibody, in Patients with Moderate to Severe Crohn's Disease.

| | |
|---|---|
| Protocol Number: | ZAF-708 |
| Phase: | II |
| Test Drug: | HuZAF (fontolizumab) |
| Indication: | Crohn's disease |
| Regulatory Status/ | Belgium, Croatia, Hungary, Russia, Slovakia, |
| Clinical Trial | United Kingdom |
| Application Location: | |
| Study Design: | Randomized, double-blind, placebo-controlled |
| | study |
| Patient Population: | Patients with moderate to severe active Crohn's |
| | disease (CD) who received prior treatment for |
| | their CD. Patients previously treated with HuZAF |
| | are excluded. |
| Inclusion Criteria: | Patients are eligible for inclusion if they are 18 to |
| | 70 years old, have received treatment for CD for |
| | at least 6 months prior to study entry, have been |
| | diagnosed as having moderate to severe active CD |
| | (CDAI score ≧250 and ≦450), agree to use |
| | adequate contraception, have a negative |
| | pregnancy test at study entry (women of child- |
| | bearing potential only), understand the purpose |
| | and risks of the study, and provide informed |
| | consent. |
| Dose Regimen/Route | Group I: A minimum of 30 patients receives a |
| of Administration: | single dose of placebo or HuZAF (4 or 10 mg/kg) |
| | given intravenously over 30 minutes. |
| | Group II: The remaining patients receive a single |
| | dose of placebo or HuZAF (4 or 10 mg/kg) given |
| | intravenously over 30 minutes once every 28 days |
| | for 2 doses. |
| Dosage Form and | Single-use glass vials containing 1.0 mL of |
| Strength/Formulation: | HuZAF at a concentration of 50 mg/mL in an |
| | isotonic buffer of histidine, glycine, and Poly- |
| | sorbate 80, at pH 6.0. Placebo consists of the |
| | formulation buffer. |
| Storage, Filtration: | The formulation contains no preservative, and |
| | must be administered without filtration. The |
| | formulation should be used within 12 hours of |
| | withdrawal and dilution into saline. |
| Duration of Treatment | Group I: Each patient has 8 visits during the |
| and Follow up: | study: one baseline visit, one treatment visit, 4 |
| | study visits, one follow-up visit, and one long- |
| | term follow-up visit, which may be in the form of |
| | a telephone call. |
| | Group II: Each patient has 9 visits during the |
| | study period: one baseline visit, 2 treatment |
| | visits, 4 study visits, one follow-up visit, and one |
| | long-term follow-up visit, which may be in the |
| | form of a telephone call. |
| | The total duration of the study for each patient |
| | is approximately 7 months. |
| Sample Size: | Up to 120 patients |
| Number of Sites: | Up to 25 investigational sites in Europe |
| Statistical Methods: | All randomized patients with an initiated infusion |
| | and valid baseline assessments are analyzed, |
| | regardless of the number of infusions that are |
| | completed (“intention-to-treat” approach). |
| | Incidences are compared using Fisher's exact test |
| | or the Cochran Mantel-Haenzel test. The changes |
| | from baseline of the continuous variables (CDAI |
| | scores and CRP values) are compared using |
| | Student's t-test or analysis of variance |
| | techniques. For time-related variables, Kaplan- |
| | Meier plots and log-rank tests may be employed. |
| | Assuming a response rate of 70% in either of the |
| | active treatment groups and a 35% response in the |
| | placebo group, the study will have an 85% power |
| | to detect a difference of 35 percentage points |
| | (at α = 0.05) for the endpoint, with 40 patients |
| | for each active treatment group and 40 patients |
| | for the placebo group. The total number of |
| | patients for the study is 120. The study is not |
| | powered to detect statistically significant |
| | differences between the 2 active dosing regimens. |
| Primary Objective(s): | 1) To evaluate the safety and tolerability of one or 2 doses of |
| | HuZAF administered to patients with moderate to severe |
| | Crohn's disease (CD) |
| | 2) To evaluate the efficacy of HuZAF as assessed by incidence |
| | of clinical responses and remissions at Day 28 following the |
| | first dose |
| Secondary | 1) To evaluate the efficacy of HuZAF in inducing responses at |
| Objective(s): | various time points |
| | 2) To evaluate the efficacy of HuZAF in inducing remissions at |
| | various time points |
| | 3) To assess the effect of HuZAF on clinical and inflammatory |
| | measures of disease (CDEIS, CDAI, CRP) |
| | 4) To assess the pharmacokinetics (PK) of HuZAF in patients |
| | with moderate to severe CD |
| | 5) To assess the incidence of anti-antibody (anti-Ab) formation |
| | in patients receiving HuZAF |
| | 6) To assess the effect of HuZAF on pharmacodynamic (PD) |
| | markers (IFN-γ-inducible chemokines/cytokines [such as IP- |
| | 10 and MIG] and CXCR3+lymphocytes) |
| Efficacy | 1) Proportion of patients who experience a clinical response on |
| Measurements: | Day 28, where clinical response is defined as a decrease of |
| | ≧100 points of the CDAI score from baseline |
| | 2) Proportion of patients who experience a clinical remission on |
| | Day 28, where clinical remission is defined as a CDAI score |
| | ≦150 |
| | 3) Clinical response and remission rates at time points in |
| | addition to Day 28 (Days 14, 42, 56, 84, and 112) |
| | 4) Duration of clinical response, where duration of clinical |
| | response is delimited by the visits at which the CDAI score |
| | improves by 100 or more points from baseline and later |
| | deteriorates to within 50 points of baseline |
| Safety Measurements: | 1) Adverse events (AE) and serious adverse events (SAE), |
| | including opportunistic infections and malignancies |
| | 2) Incidence of anti-Ab formation |
| | 3) Changes in physical exam findings, vital signs, and |
| | laboratory values |
| Pharmacodynamic | Endoscopy may be performed at selected sites. CDEIS scores |
| Measurements | are used to assess endoscopic improvement of CD. Exploratory |
| | PD markers are assessed at selected sites. The analysis of IFN-γ- |
| | inducible chemokines/cytokines in the serum (such as IP-10 and |
| | MIG) and CXCR3+ lymphocytes in the peripheral blood is |
| | performed. |
| Pharmacokinetic | Blood samples are collected from patients for PK analysis at |
| Measurements | selected sites. |
| Anti-Ab Assessments | Blood samples are collected from all patients for measurement of |
| | anti-Ab levels. |

Although the invention has been described with reference to the presently preferred embodiments, it should be understood that various modifications maybe made without departing from the spirit of the invention.

All publications, patents, patent applications, and web sites are herein incorporated by reference in their entirety to the same extent as if each individual patent, patent application, or web site was specifically and individually indicated to be incorporated by reference in its entirety.

TABLE 1
Crohn's Disease Activity Index (CDAI)

| DISEASE ACTIVITY INDEX | | |
|---|---|---|
| SUBTOTAL | SUM | X FACTOR |
| Total number of liquid or very soft stools | | |
| in the previous 7 days or, for stoma patients, | | |
| total number of bags emptied: | | × 2 = |
| Sum abdominal pain/cramps ratings | | |
| (total for previous 7 days): | | × 5 = |

| | |
|---|---|
| 0 = none | 2 = moderate |
| 1 = mild | 3 = severe |

| | |
|---|---|
| General well being | |
| (total for previous 7 days): | × 7 = |

| | |
|---|---|
| 0 = generally well | 3 = very poor |
| 1 = slightly under par | 4 = terrible |
| 2 = poor | |

| | |
|---|---|
| Categories currently present and presumed | |
| to be related to Crohn's disease: 0 = no; 1 = yes | |
| □ =arthritis/arthralgia | × 20 = |
| □ =iritis/uveitis | × 20 = |
| □ =erythema nodosum/pyoderma | × 20 = |
| gangrenosum/aphthous stomatitis | |
| □ =anal fissure, fistula or abscess | × 20 = |
| □ other fistula | × 20 = |
| □ fever over 37.8 0C during the | |
| previous 7 days | |
| During the previous 7 days has patient received | × 30 = |
| Anti-diarrhea/opiate therapy at least once: | |

| | |
|---|---|
| 0 = no | 1 = yes |

| | |
|---|---|
| Abdominal mass: | × 10 = |

| | | |
|---|---|---|
| 0 = none | 2 = questionable | 5 = definite |

| Hematocrit: | | × 6 = |
|---|---|---|
| Males: (47-Hct) = SUM | (add or subtract by sign) | |
| Females: (42-Hct) = SUM | | |
| (Standard Weight) - (Actual Body Weight) × 100 | | × 1 = |
| Standard Weight | (add or subtract by sign, round to 3 decimal places) | |
| | | TOTAL = |
| | | (round total to integer) |

TABLE 2
Psoriasis Area and Severity Index (PASI)

```
A. Anatomic Sites
  To calculate a subject's PASI score, the following 4 anatomic sites are assessed:
    Head (h)
    Upper extremities (u)
    Trunk (t)
    Lower extremities (l)
  These sites roughly correspond to 10, 20, 30, and 40% of the
  body surface area (BSA), respectively.
B. PASI Score Calculation
  The PASI score is calculated using the following formula:
```

PASI = 0.1(Eₕ + Iₕ + Dₕ)Aₕ + 0.2 (Eᵤ + Iᵤ + Dᵤ)Aᵤ + 0.3(Eₜ + Iₜ + Dt)Aₜ +

0.4(Eₗ + Iₗ + Dₗ)Aₗ,

```
  where E = erythema, I = induration, D = desquamation, and A = area
  E, I, and D are assessed according to a 5-point scale:
  0 = no symptoms
  1 = slight
  2 = moderate
  3 = marked
  4 = very marked
  “A” is assigned a numerical value based on the extent of lesions in a given site:
  1 = <10%
  2 = 10 to 29%
  3 = 30 to 49%
  4 = 50 to 69%
  5 = 70 to 89%
  6 = 90 to 100%
  The PASI varies in steps of 0.1 units from 0.0 to 72.0. The highest
  score represents complete
erythroderma of the severest possible degree.
```

TABLE 3
Crohn's Disease Endoscopic Index of Severity (CDEIS)

For the endoscopic findings, segmental data will be collected for the following 5 segments:
rectum, sigmoid and left colon, transverse colon, right colon (and cecum), and ileum.
The following will be collected per segment:
1) Nature of elementary mucosal lesions

| LESIONS | DEFINITIONS OR SPECIFICATIONS |
|---|---|
| 1 = Pseudopolyp | |
| 2 = Healed ulceration | Whitish area with a “ground glass” appearance |
| 3 = Frank erythema (plaques, bands, or diffuse) | Slight or moderate erythema should be neglected |
| 4 = Frankly swollen mucosa | Slight or moderate mucosal swelling should be |
| | neglected |
| 5 = Aphtoid ulceration | Defined as a tiny (2 to 3 mm), raide or flat red lesions |
| | with a white center |
| 6 = Superficial or shallow ulceration | Defined as any ulceration which was neither aphtoid nor |
| | deep |
| 7 = Deep ulceration | Only frankly deep ulceration should be recorded under |
| | this heading |
| 8 = Non-ulcerated stenosis | Should be impossible or difficult to pass with an adult |
| | endoscopic |
| 9 = Ulcerated stenosis | Should be impossible or difficult to pass with an adult |
| | endoscopic |

2) The percentage of the surface involved by the disease, taking into account (A) any of the nine lesions
listed above and (B) by ulcerations only (included are lesion numbers 5, 6, 7, and 9).
This will be done by putting a cross on two, 10-cm linear analog scales.

CDEIS

Date of Colonoscopy (D-M-Y):
If not done, record reason on Comment Page
Check all that apply, with the exception that per segment choice 0 may not be combined with
choices 1 to 9.
Refer to page 54 for description of lesions.

| | | Sigmoid and | Transverse | Right Colon and | |
|---|---|---|---|---|---|
| Rectum | Left Colon | Colon | Cecum | Ileum | |
| Not Evaluated | Reasons: | Reasons: | Reasons: | Reasons: | Reasons: |
| (check one | □ Technically | □ Technically | □ Technically | □ Technically | □ Technically |
| only) | impossible | impossible | impossible | impossible | impossible |
| | □ Pt. | Pt. Intolerance | Pt. Intolerance | Pt. Intolerance | Pt. Intolerance |
| | Intolerance | | | | |
| 0 = No | | | | | |
| abnormalities | | | | | |
| 1 = | | | | | |
| Pseudopolyp | | | | | |
| 2 = Healed | | | | | |
| ulceration | | | | | |
| 3 = Frank | | | | | |
| erythema | | | | | |
| (plaques, | | | | | |
| bands, diffuse) | | | | | |
| 4 = Frankly | | | | | |
| swollen | | | | | |
| mucosa | | | | | |
| 5 = Aphtoid | | | | | |
| ulceration | | | | | |
| 6 = Superficial | | | | | |
| or shallow | | | | | |
| ulceration | | | | | |
| 7 = Deep | | | | | |
| ulceration | | | | | |
| 8 = Non- | | | | | |
| ulcerated | | | | | |
| stenosis | | | | | |
| 9 = Ulcerated | | | | | |
| stenosis | | | | | |
| % Segment | | | | | |
| involved (all | | | | | |
| lesions | | | | | |
| % Segment | | | | | |
| involved (all | | | | | |
| ulcerations) | | | | | |

Indicate by putting a cross on the bar, the percentage of the surface involved by the
disease, taking into account (A) any of the nine lesions and (B) by ulcerations only.

| Segment | 0% | 100% |
|---|---|---|
| Rectum | Lesions | A |
| | Ulcerations | B |
| Sigmoid and left colon | Lesions | A |
| | Ulcerations | B |
| Right Colon and cecum | Lesions | A |
| | Ulcerations | B |
| Ileum | Lesions | A |
| | Ulcerations | B |

Signature of the evaluator performing the colonscopy:

TABLE 5
Summary of PASI Scores
Patients Who Received HuZAF (N = 16)

| | | | | |
|---|---|---|---|---|
| 0.1 mg/kg | 1.0 mg/kg | 3.0 mg/kg | 10.0 mg/kg | Total |
| n = 4 (25%) | n = 4 (25%) | n = 4 (25%) | n = 4 (25%) | N = 16 (100%) |

Baseline

| | | | | | | |
|---|---|---|---|---|---|---|
| PASI Scores | N | 4 | 4 | 4 | 4 | 16 |
| | Mean (SD) | 18.0 (8.4) | 9.0 (4.8) | 9.6 (3.8) | 15.4 (5.1) | 13.0 (6.5) |
| | Median | 17.0 | 8.3 | 8.0 | 16.7 | 11.0 |
| | (Min, Max) | (10.5, 27.6) | (4.3, 15.0) | (7.0, 15.3) | (8.5, 19.5) | (4.3, 27.6) |

Visit 6 (D15)

| | | | | | | |
|---|---|---|---|---|---|---|
| PASI Scores | N | 4 | 4 | 4 | 4 | 16 |
| | Mean (SD) | 17.2 (8.2) | 8.6 (4.7) | 5.7 (1.6) | 11.6 (6.2) | 10.8 (6.8) |
| | Median | 15.4 | 8.1 | 5.9 | 11.1 | 9.9 |
| | (Min, Max) | (10.5, 27.6) | (3.7, 14.4) | (3.6, 7.4) | (4.7, 19.5) | (3.6, 27.6) |

% Change from Baseline

| | | | | | |
|---|---|---|---|---|---|
| >= 50% Imp | 0 (0.0%) | 0 (0.0%) | 1 (25.0%) | 0 (0.0%) | 1 (6.3%) |
| >= 75% Imp | 0 (0.0%) | 0 (0.0%) | 1 (25.0%) | 0 (0.0%) | 1 (6.3%) |
| N | 4 | 4 | 4 | 4 | 16 |
| Mean (SD) | −4.3 (5.4) | −5.4 (6.0) | −31.9 (30.7) | −26.8 (23.8) | −17.1 (21.9) |
| Median | −3.1 | −3.9 | −22.3 | −29.2 | −8.7 |
| (Min, Max) | (−11.1, 0.0) | (−14.0, 0.0) | (−76.5, −6.3) | (−48.9, 0.0) | (−76.5, 0.0) |

Visit 7 (D29)

| | | | | | | |
|---|---|---|---|---|---|---|
| PASI Scores | N | 3 | 4 | 4 | 4 | 15 |
| | Mean (SD) | 12.3 (4.1) | 8.3 (4.5) | 6.6 (1.6) | 11.3 (5.6) | 9.4 (4.4) |
| | Median | 14.0 | 6.7 | 6.3 | 9.5 | 7.6 |
| | (Min, Max) | (7.6, 15.3) | (4.9, 14.8) | (5.1, 8.5) | (6.8, 19.5) | (4.9, 19.5) |

% Change from Baseline

| | | | | | |
|---|---|---|---|---|---|
| >= 50% Imp | 0 (0.0%) | 0 (0.0%) | 1 (25.0%) | 0 (0.0%) | 1 (6.3%) |
| >= 75% Imp | 0 (0.0%) | 0 (0.0%) | 0 (0.0%) | 0 (0.0%) | 0 (0.0%) |
| N | 3 | 4 | 4 | 4 | 15 |
| Mean (SD) | −8.5 (47.0) | −4.6 (19.0) | −21.9 (37.3) | −26.1 (21.1) | −15.7 (29.6) |
| Median | −33.3 | −0.7 | −21.1 | −27.8 | −20.0 |
| (Min, Max) | (−37.8, 45.7) | (−31.1, 14.0) | (−66.7, 21.4) | (−48.9, 0.0) | (−66.7, 45.7) |

Visit 8 (D57)

| | | | | | | |
|---|---|---|---|---|---|---|
| PASI Scores | N | 2 | 4 | 3 | 2 | 11 |
| | Mean (SD) | 15.2 (3.6) | 8.9 (3.4) | 6.9 (1.8) | 14.8 (15.1) | 10.5 (6.4) |
| | Median | 15.2 | 7.6 | 7.3 | 14.8 | 8.2 |
| | (Min, Max) | (12.6, 17.7) | (6.6, 13.8) | (4.9, 8.4) | (4.1, 25.5) | (4.1, 25.5) |

% Change from Baseline

| | | | | | |
|---|---|---|---|---|---|
| >= 50% Imp | 0 (0.0%) | 0 (0.0%) | 0 (0.0%) | 1 (25.0%) | 1 (6.3%) |
| >= 75% Imp | 0 (0.0%) | 0 (0.0%) | 0 (0.0%) | 0 (0.0%) | 0 (0.0%) |
| N | 2 | 4 | 3 | 2 | 11 |
| Mean (SD) | 39.5 (41.0) | 11.8 (40.5) | −9.0 (29.8) | −10.5 (58.4) | 7.1 (39.2) |
| Median | 39.5 | 14.3 | −7.6 | −10.5 | 10.5 |
| (Min, Max) | (10.5, 68.6) | (−34.9, 53.5) | (−39.5, 20.0) | (−51.8, 30.8) | (−51.8, 68.6) |

Visit 9 (D85)

| | | | | | | |
|---|---|---|---|---|---|---|
| PASI Scores | N | 1 | 3 | 3 | 2 | 9 |
| | Mean (SD) | 6.0 (—) | 9.2 (5.8) | 6.4 (0.9) | 19.6 (20.3) | 10.2 (9.5) |
| | Median | 6.0 | 5.9 | 6.7 | 19.6 | 6.0 |
| | (Min, Max) | (6.0, 6.0) | (5.8, 15.9) | (5.3, 7.1) | (5.2, 33.9) | (5.2, 33.9) |

% Change from Baseline

| | | | | | |
|---|---|---|---|---|---|
| >= 50% Imp | 0 (0.0%) | 0 (0.0%) | 0 (0.0%) | 0 (0.0%) | 0 (0.0%) |
| >= 75% Imp | 0 (0.0%) | 0 (0.0%) | 0 (0.0%) | 0 (0.0%) | 0 (0.0%) |
| N | 1 | 3 | 3 | 2 | 9 |
| Mean (SD) | −42.9 (—) | 52.3 (106) | −16.1 (18.0) | 17.5 (79.7) | 11.2 (70.5) |
| Median | −42.9 | 37.2 | −15.2 | 17.5 | −15.2 |
| (Min, Max) | (−42.9, | (−45.3, | (−34.6, 1.4) | (−38.8, 73.8) | (−45.3, 165.0) |
| | −42.9) | 165.0) | | | |

End of Double-Blind Treatment And F/U Period*

| | | | | | | |
|---|---|---|---|---|---|---|
| PASI Scores | N | 4 | 4 | 4 | 4 | 16 |
| | Mean (SD) | 15.1 (9.1) | 10.4 (5.3) | 6.5 (0.8) | 15.7 (12.7) | 11.9 (8.3) |
| | Median | 13.3 | 9.9 | 6.8 | 11.8 | 8.3 |
| | (Min, Max) | (6.0, 27.6) | (5.8, 15.9) | (5.3, 7.1) | (5.2, 33.9) | (5.2, 33.9) |

% Change from Baseline

| | | | | | |
|---|---|---|---|---|---|
| >= 50% Imp | 0 (0.0%) | 0 (0.0%) | 1 (25.0%) | 0 (0.0%) | 1 (6.3%) |
| >= 75% Imp | 0 (0.0%) | 0 (0.0%) | 0 (0.0%) | 0 (0.0%) | 0 (0.0%) |
| N | 4 | 4 | 4 | 4 | 16 |
| Mean (SD) | −17.5 (26.7) | 37.2 (91.6) | −25.8 (24.3) | −6.3 (53.8) | −3.1 (56.1) |
| Median | −18.9 | 14.6 | −24.9 | −30.0 | −19.8 |
| (Min, Max) | (−42.9, 10.5) | (−45.3, | (−54.9, 1.4) | (−38.8, 73.8) | (−54.9, 165.0) |
| | | 165.0) | | | |

Note:
Data collected after prohibited medication or in open label phase are excluded
% Change from baseline = 100* (PASI score − baseline score)/(baseline score), negative values indicate improvement
*= Last observation carried forward for patients who terminated early.

We claim:

1. A method for treating Crohn's disease in a subject in need of such a treatment, comprising administering to said subject a therapeutically effective amount of an antibody against interferon γ.

2. The method according to claim 1, wherein said treatment reduces severity of Crohn's disease.

3. The method according to claim 1, wherein said treatment reduces CDAI score of said subject.

4. The method according to claim 3, wherein said CDAI score is reduced by more than 70 points.

5. The method according to claim 3, wherein said CDAI score is reduced by more than 100 points.

6. The method according to claim 1, wherein said treatment causes a remission of Crohn's disease indicated as CDAI score reduced to less than 150 points.

7. The method according to claim 1, wherein said antibody neutralizes interferon γ.

8. The method according to claim 1, wherein said antibody is a humanized antibody.

9. The method according to claim 8, wherein said humanized antibody is HuZAF.

10. The method according to claim 1, wherein said antibody binds to the same epitope as HuZAF.

11. The method according to claim 1, wherein said antibody is a chimeric antibody or fully human antibody.

12. The method according to claim 1, wherein said antibody has a binding affinity for human interferon γ of at least 10⁸ M⁻¹.

13. The method according to claim 12, wherein said antibody has a binding affinity for human interferon γ of at least 10⁹ M⁻¹.

14. The method according to claim 1, wherein the antibody is administered intravenously, intramuscularly, or subcutaneously.

15. The method according to claim 1, wherein the subject is a human.

16. The method according to claim 1, wherein said therapeutically effective amount is from 0.01 mg/kg to 100 mg/kg.

17. The method according to claim 16, wherein said therapeutically effective amount is from 0.1 mg/kg to 10 mg/kg.

18. The method according to claim 1, wherein said antibody is administered with a frequency from daily to every 6 month.

19. The method according to claim 18, wherein said antibody is administered daily, 2 or 3 times a week, biweekly, or monthly, every 6 weeks, or every 2 or 3 months.

20. The method according to claim 1, wherein said antibody is administered to said subject at least once.

21. The method according to claim 15, wherein said administering comprises administering of a first dose of said antibody and a second dose of said antibody.

22. The method according to claim 21, wherein said second dose is administered later than the first dose.

23. The method according to claim 22, wherein said second dose is less than said first dose.

24. The method according to claim 23, wherein said second dose is 50% of said first dose.

25. The method according to claim 21, wherein said first dose is 0.1, 1, 4, or 10 mg/kg.

26. The method according to claim 21, wherein said first dose is about 1.0 mg/kg and said second is about 0.1 mg/kg.

27. The method according to claim 21, wherein said first dose is about 1.0 mg/kg and said second dose is about 1.0 mg/kg.

28. The method according to claim 21, wherein said first dose is about 4.0 mg/kg and said second dose is about 0.1 mg/kg.

29. The method according to claim 21, wherein said first dose is about 4.0 mg/kg and said second dose is about 1.0 mg/kg.

30. The method according to claim 22, wherein said first dose is administered through intravenous infusion and said second dose is administered through subcutaneous injection.

31. The method according to claim 22, wherein the administering of said second dose repeats at least once.

32. The method according to claim 31, said antibody is administered daily, 2 or 3 times a week, biweekly, or monthly, every 6 weeks, or every 2 or 3 months.

33. The method according to claim 32, wherein said second dose is administered every 4 weeks.

34. A method for treating psoriasis in a subject in need of such a treatment, comprising administering to said subject a therapeutically effective amount of an antibody against interferon γ.

35. The method according to claim 34, whereby said treatment reduces PSAI score of said subject.

36. The method according to claim 35, wherein the PSAI score is reduced by at least 50%.

37. The method according to claim 36, wherein the PSAI score is reduced by at least 75%.

38. The method according to claim 34, wherein at least 25% of patients receiving said treatment have a reduction of PSAI score by at least 50%.

39. The method according to claim 34, wherein the subject is a human.

40. The method according to claim 34, wherein said antibody neutralizes interferon γ.

41. The method according to claim 34, wherein said antibody is a humanized antibody.

42. The method according to claim 41, wherein said humanized antibody is HuZAF.

43. The method according to claim 34, wherein said antibody binds to the same epitope as HuZAF.

44. The method according to claim 34, wherein said antibody is a chimeric antibody or fully human antibody.

45. The method according to claim 34, wherein said antibody has a binding affinity for human interferon γ of at least 10⁸ M⁻¹.

46. The method according to claim 45, wherein said antibody has a binding affinity for human interferon γ of at least 10⁹ M⁻¹.

47. The method according to claim 34, wherein the antibody is administered intravenously, intramuscularly, or subcutaneously.

48. The method according to claim 34, wherein said therapeutically effective amount is from 0.01 mg/kg to 100 mg/kg.

49. The method according to claim 48, wherein said therapeutically effective amount is from 0.1 mg/kg to 10 mg/kg.

50. The method according to claim 34, wherein said antibody is administered with a frequency from daily to every 6 month.

51. The method according to claim 50, wherein said antibody is administered daily, 2 or 3 times a week, biweekly, or monthly, every 6 weeks, or every 2 or 3 months.

52. The method according to claim 50, wherein said antibody is administered to said subject at least once.
